(19) United States
(12) Patent Application Publication
(10) Pub. No.: US 2023/0051563 A1
(43) Pub. Date: Feb. 16, 2023

(54) NON-ELICITED DEDIFFERENTIATED LAVANDULA ANGUSTIFOLIA PLANT CELLS, EXTRACTS THEREOF AND COSMETIC USES THEREOF

(71) Applicant: L'OREAL, Paris (FR)

(72) Inventors: Cosima DUFOUR-SCHROIF, Aulnay-Sous-Bois (FR); Franck JUCHAUX, Aulnay-Sous-Bois (FR); Pascal HILAIRE, Aulnay-Sous-Bois (FR); Catherine BESOMBES, Aulnay-Sous-Bois (FR); Emilie GUENAULT, Tours Cedex 2 (FR)

(21) Appl. No.: 17/786,574

(22) PCT Filed: Dec. 16, 2020
(86) PCT No.: PCT/EP2020/086559

§ 371 Date: Jun. 17, 2022

(30) Foreign Application Priority Data
Dec. 19, 2019 (FR) FR1914938

(51) Int. Cl. A61K 8/9789; C12N 5/04; A61Q 19/00; C12N 5/00

(52) U.S. Cl. CPC A61K 8/9789; C12N 5/04; A61Q 19/00; A61Q 19/007; C12N 5/0025

(57) ABSTRACT

The present invention relates to non-elicited dedifferentiated plant cells of a plant of the species Lavandula angustifolia, or extracts thereof, and also to the cosmetic composition comprising same, to the uses thereof and to cosmetic treatment processes comprising the application of said composition to the skin for improving and/or reinforcing the barrier function, and also for improving the moisturization of the skin.

The present invention relates to the field of skincare.

The invention relates to non-elicited dedifferentiated plant cells of a plant of the species Lavandula angustifolia, to extracts thereof and also to a cosmetic composition comprising same.

The present invention also relates to a cosmetic process for treating the skin, which is intended to improve the barrier function, comprising at least one step consisting in applying to the skin at least one composition as defined above.

In particular, the composition of the invention is intended to improve and/or reinforce the skin's barrier function. The invention moreover finds a use in skin moisturization, in improving the suppleness of the skin, and in improving and/or reducing the skin's microrelief. The invention also finds a use in treating dry skin.

Human skin is made up of two compartments, namely a deep compartment, the dermis, and a superficial compartment, the epidermis.

The dermis provides the epidermis with a solid support. It is also its nourishing element. It mainly consists of fibroblasts and an extracellular matrix, which is itself composed mainly of collagen, elastin and a substance known as ground substance, these components being synthesized by the fibroblast. Leukocytes, mast cells and tissue macrophages are also found therein. It also contains blood vessels and nerve fibres.

The epidermis is in contact with the external environment.

Natural human epidermis is composed mainly of three types of cells, namely keratinocytes, which form the vast majority, melanocytes and Langerhans cells.

The cells constituting the epidermis are delimited by an intercellular lipid domain.

Each of these cell types contributes, by virtue of its intrinsic functions, toward the essential role played in the body by the skin. In particular, the keratinocytes undergo a continuous and oriented maturation process which, from the keratinocytes that are present in the basal layer of the epidermis, results in the formation of corneocytes, which are totally keratinized dead cells consisting of keratinocytes in the terminal stage of their differentiation.

In the course of differentiation, the phospholipids, the role of which consists in producing the fluid structure of the cell membranes of the live layers of the epidermis, are gradually replaced with a mixture predominantly composed of fatty acids, cholesterol and sphingolipids (ceramides). These lipids, which are organized as specific lamellar liquid crystal phases, form the intracellular cement of the stratum corneum and are essential for the exchanges of water and the barrier function of the epidermis. Thus, the lamellar structure of the lipids of the lipid domain of the epidermis and the corneocytes participate in the epidermal barrier function.

The skin thus constitutes a barrier against external attack, notably chemical, mechanical or infectious attack, and, in this respect, a certain number of defense reactions against environmental factors (climate, ultraviolet rays, tobacco, etc.) and/or xenobiotic factors, for instance microorganisms, take place thereon.

This property, known as the barrier function, is primarily ensured by the uppermost layer of the epidermis, namely the cornified layer known as the stratum corneum.

It is clear that the quality and the equilibrium of the cutaneous and mucous membrane barrier is dependent on complex endogenous biological mechanisms involving numerous growth factors, adhesion molecules, hormones and enzymes of lipid metabolism.

Thus, impairment of the skin barrier may arise in the presence of external attacking factors such as irritants (detergents. acids, bases, oxidizing agents, reducing agents, concentrated solvents, gases or toxic fumes), mechanical stresses (friction, impacts, abrasion, tearing of the surface, projection of dusts or particles, shaving or hair removal), thermal or climatic imbalances (cold, dryness, UV radiation), xenobiotics (undesirable microorganisms, allergens) or internal attacking factors such as psychological stress.

The following people may be more particularly concerned by this impairment of the barrier function by external attack:
people with “fragile” or “delicate” and vulnerable skin which becomes rapidly imbalanced during large-amplitude variations in temperature or relative humidity (for example in the case of baby skin);
people with “embrittled” skin, notably including
people whose protective hydro-lipid film composed of sweat, sebum and natural moisturizing factors becomes diminished, as is the case for elderly people over 60 years old and notably in the case of the very elderly (at least 75 years old);
people for whom the composition of the hydro-lipid film is modified;
people who have a lowered reactivity threshold due to neurogenic hyperactivity; these skin types will thus present these sensations and clinical signs much more quickly and frequently than other skin types: these are people with sensitive skin.

Mention may also not be made of people with “attacked” skin, for example shaved skin.

Impairment of the cutaneous barrier function may notably be reflected by a moisturization disorder, loss of suppleness of the skin, impairment of the radiance of the complexion and the appearance of roughness on the skin, or impairment of its microrelief.

It is then appropriate to seek to increase the epidermal differentiation in order to reinforce the skin's barrier function.

In particular, it is thus sought to improve and/or reinforce the cutaneous barrier function in order:
to overcome skin moisturization problems, notably of the mucous membranes, and notably to treat dry skin,
to improve the suppleness of the skin,
to maintain and/or improve the radiance of the complexion,
to prevent and/or treat skin roughness or impairment of the skin's microrelief.

In order to thwart an imbalance in the barrier function, particular attention should be paid to active agents of natural origin, notably dedifferentiated plant cells and also extracts thereof.

Dedifferentiated plant cells arose from the work by Haberland in 1902. Over the last 40 years, plant cell cultures have been used for the production of metabolites of interest or for the multiplication of plants that are exactly the same (somatic embryogenesis). This plant biotechnology is based on the concept of cell totipotency: “any plant cell is capable of dedifferentiating and of regenerating another individual identical to that from which it is derived”. A dedifferentiated plant cell is a plant cell originating from an organ (leaf, stem, root, petal, etc.) which has been placed in culture, and which loses its organ specificity, notably its leaf, stem, root or petal specificity, and once again becomes potentially capable of generating the whole plant.

An undifferentiated plant cell is the equivalent of a real plant stem cell, derived from meristematic plant cells, and not having an organ-specific biological past.

For example, WO 2009/151302 and KR 2009-0118877 describe antiaging or antioxidant compositions containing undifferentiated plant cells derived from the cambium of Panax ginseng or from a plant of the Taxus genus.

It is known practice from the prior art to use dedifferentiated plant cells in cosmetics, mainly in the form of extracts, for the properties that have been acknowledged for said plant cells.

EP1485064 notably describes cosmetic compositions comprising a ground material of elicited dedifferentiated plant cells from Lavandula angustifolia and Vitex negundo which have antioxidant properties.

Thus, there is a need to identify novel technical solutions for improving and/or reinforcing the skin's barrier function.

In particular, there is a need to propose novel active agents for improving and/or reinforcing the protection of the skin against external attack, for improving the moisturization of the skin, the suppleness of the skin and the radiance of the complexion and/or for reducing the roughness or microrelief of the skin.

The object of the present invention is notably to meet these needs.

Specifically, the inventors have now demonstrated that non-elicited dedifferentiated plant cells of a plant of the species Lavandula angustifolia, or extracts thereof. can improve and/or reinforce the skin's barrier function.

Notably, the absence of elicitation of the dedifferentiated plant cells of Lavandula angustifolia makes it possible to obtain an increase in the expression of barrier function and moisturization markers such as those responsible for the assembly of the cornified layer (SPRR1A, CNFN), for keratinocyte differentiation (AQP3); or those responsible for the synthesis of the glycosaminoglycans GAGs (HAS3) and for epidermal renewal (HBEGF), as shown in comparative example 3b below.

To the inventors' knowledge, no document makes reference to the production of a non-elicited dedifferentiated cell line of a plant of the species Lavandula angustifolia, or of extracts thereof, which have the specific cosmetic properties described in the present description.

According to one of its first aspects, the present invention relates to non-elicited dedifferentiated plant cells of a plant of the species Lavandula angustifolia, or extracts thereof.

A second subject of the present invention relates to a cosmetic composition comprising, in a physiologically acceptable medium, said cells and/or extracts according to the invention.

The present invention also relates to the cosmetic use of non-elicited dedifferentiated plant cells of a plant of the species Lavandula angustifolia, or extracts thereof, for improving and/or reinforcing the skin's barrier function.

According to another of its aspects, the invention also relates to the cosmetic use of non-elicited dedifferentiated plant cells of a plant of the species Lavandula angustifolia, or extracts thereof, for improving and/or reinforcing the protection of the skin against external attack.

The invention moreover relates to the cosmetic use of non-elicited dedifferentiated plant cells of a plant of the species Lavandula angustifolia, or extracts thereof, for improving the moisturization of the skin, for preventing and/or treating roughness or the microrelief and/or for improving the radiance of the complexion and/or for improving the suppleness of the skin.

In, addition, the present invention relates to the cosmetic use of non-elicited dedifferentiated plant cells of a plant of the species Lavandula angustifolia, or extracts thereof, for preventing and/or treating the cosmetic signs of skin dryness.

Another subject of the present invention is a cosmetic skin treatment process, comprising the application to the skin of the composition according to the invention for improving and/or reinforcing the cutaneous barrier function of the skin.

The invention also relates to a cosmetic skin treatment process, comprising the application to the skin of the composition according to the invention for improving and/or reinforcing the protection of the skin against external attack.

The invention also relates to a cosmetic skin treatment process, comprising the application to the skin of the composition according to the invention for improving the moisturization of the skin, for preventing and/or treating roughness or the microrelief and/or for improving the radiance of the complexion and/or for improving the suppleness of the skin.

The invention also relates to a cosmetic process for treating dry skin, comprising the application to dry skin of the composition according to the invention for treating the cosmetic signs of skin dryness.

The process according to the invention is notably intended for people with skin dryness, irrespective of the age or type of the person's skin or the cause of the dryness.

According to another embodiment, said composition may be intended for improving and/or reinforcing the barrier function of a skin chosen from fragile skin, embrittled skin, attacked skin and/or sensitive skin.

In the context of the invention, the composition may be used for application to healthy skin, which is subjected or which may be subjected to external attack as recalled above. In other particular cases, the composition of the invention may be applied to the skin when it presents clinical signs of cutaneous barrier deficiency.

### DEFINITIONS

The term “cosmetic composition” means a composition comprising a physiologically acceptable medium, i.e. a medium that is compatible with the skin.

The term “skin” refers to all of the skin of the body, and preferably the skin of the face, neckline, neck, arms and forearms, or even more preferably the skin of the face. notably of the forehead, nose, cheeks, chin and area around the eyes.

The term “non-elicited cells” means cells which have not undergone elicitation.

The term “elicitation” means herein the induction, by means of an exogenous elicitor, in another organism or cell, of a sparingly expressed metabolic pathway or the wakening, in another organism or cell, of silenced metabolic pathways.

The term“elicitors” means herein molecules or organisms that are capable of inducing, in another organism, a sparingly expressed metabolic pathway or of wakening, in another organism, silenced metabolic pathways. A large number of elicitors are known to those skilled in the art and include biotic elicitors, such as jasmonate and derivatives thereof, and abiotic elicitors such as the temperature, the pH, UV, gases such as CO2, or an osmotic shock.

The term “process not comprising an elicitation step” means a process in which the dedifferentiated cells are not placed in contact with an elicitor as defined above.

The term “placing in contact” means herein incubating, in the same culture medium, the dedifferentiated plant cells and an eliciting agent.

The term “cosmetic signs of skin dryness” means the sensations of tautness and/or tension of the skin, the appearance of squamae on the skin, and/or the appearance of rough-feeling skin.

### DETAILED DESCRIPTION

### Non-Elicited Dedifferentiated Lavandula anqustifolia Plant Cells, Extracts Thereof

For the purposes of the invention, the term “dedifferentiated plant cell” means any cell strain derived from organs of a plant of the Lavandula angustifolia species and obtained under specific in vitro culture conditions, no longer exhibiting any specialization character and capable, under the effect of induction, of any differentiation in accordance with its genome and of generating by itself a whole plant of a plant from which it originates. Such cells are capable of living by themselves and not in a dependency relationship with other cells.

Dedifferentiated plant cells are distinct from undifferentiated plant cells which naturally exist in plants.

For the purposes of the invention, the term “dedifferentiated plant cell” means a strain obtained by in vitro cultivation, derived from organs of a plant of the species Lavandula angustifolia, which is capable of differentiating, and/or of acquiring new characteristics of a specialized cell, under the effect of an induction into any cell type (totipotent) or into several cell types (pluripotent), in particular embryogenic or meristematic cells.

Under normal conditions, plant cells express about 20% of their genome, the remaining 80% being expressed only in response to particular environmental conditions. The in vitro cultivation of these cells under particular culture conditions makes it possible to “reprogram” the cells and thus to access a part of this genome not expressed in the whole plant. Some compounds, which are difficult to obtain by extraction from plants, become more accessible in cell cultures.

Thus, advantageously, the dedifferentiated plant cells of the invention make it possible to access novel compounds not present in the whole plant, or to significantly increase the expression of molecules that are known but rare in the whole plant.

The present invention also relates to non-elicited dedifferentiated cells of a plant of the species Lavandula angustifolia, said dedifferentiated cells being obtained by means of the process detailed in the present description, including in the examples.

The inventors have shown that the process according to the invention makes it possible to obtain dedifferentiated cell lines of a plant of the species Lavandula angustifolia, it being possible for the cells in the lines to be cultivated in the form of dedifferentiated cells over a very long period of time, without any detectable modification of their morphology, and without any detectable modification of their properties, in particular without any detectable modification of their properties with regard to the barrier function and moisturization.

Non-elicited dedifferentiated plant cells of the invention may be obtained from any plant part of the species Lavandula angustifolia or from cells of said plants.

The term “plant part(s)” means either one or more whole organs of the plant, for instance the leaves, the stalks, the flowers, the petals, the sepals, the seeds or the roots, or one or more fragments of said plant organ(s) cultivated in vivo or wild. Use may thus be made of one or more leaves or one or more leaf fragments of the plant Lavandula angustifolia for producing non-elicited dedifferentiated plant cells of the invention.

The term “in vivo cultivation” means any cultivation of conventional type, i.e. in soil in the open air or in a greenhouse, or alternatively out of the soil.

The term “in vitro cultivation” means all the techniques known to those skilled in the art for artificially obtaining a plant or a plant part, in a reproducible manner.

Preferentially according to the invention, a plant obtained from in vivo cultivation and more preferentially a plant part obtained from in vivo cultivation is used.

Preferably, non-elicited dedifferentiated plant cells of the invention are obtained from at least one leaf or leaf part of a plant of the species Lavandula angustifolia.

More preferably, the non-elicited dedifferentiated plant cells of the invention are obtained from a plant of the white variety of Lavandula angustifolia. This variety is notably registered in the French Pharmacopea.

The plants of the white variety of Lavandula angustifolia can be chosen from Lavandula angustifolia «Hidcote White», Lavandula angustifolia «Silbermöwe», Lavandula angustifolia «Alba», Lavandula angustifolia «Arctic Snow» sold by the supplier le jardin du pic vert, or white Lavandula angustifolia cultivated in Drôme, France.

In a very preferred embodiment, the white variety of Lavandula angustifolia originates from Drôme (France).

Entirely preferably, the non-elicited dedifferentiated plant cells according to the invention are obtained using, as starting product, leaves of a plant of the white variety of Lavandula angustifolia.

Advantageously, a culture medium that is suitable for obtaining non-elicited dedifferentiated plant cells of the invention comprises hormones that are naturally present in the plants, said culture medium not comprising an elicitor.

According to a preferential embodiment, the non-elicited dedifferentiated cells of a plant of the species Lavandula angustifolia are obtained by means of a process comprising the following steps:

i. providing one or more plant parts, in particular one or more whole leaves or one or more leaf fragments of the species Lavandula angustifolia;
ii. cultivating said plant part(s) provided in step i. in a culture medium comprising at least one plant hormone, so as to generate dedifferentiated cells; and
iii. recovering the dedifferentiated cells obtained at the end of step ii.;
iv. optionally, extracting the dedifferentiated cells recovered in step iii.; said process not comprising a step of eliciting said dedifferentiated cells.

Preferentially, in step ii., the plant part(s), in particular the whole leaf (leaves) or the leaf fragment(s), are cultivated in anexic form (freed of any biological contaminant).

In step ii. of the process, said plant part(s) are cultivated in a suitable culture medium, which comprises at least one plant hormone also known as a phytohormone. In certain embodiments, said culture medium comprises a plurality of plant hormones, for example two or three plant hormones.

The phytohormones contained in said culture medium may be chosen from auxins, cytokinins, gibberellins, and mixtures thereof.

The auxins that are suitable for use in the invention may be chosen from IAA (indole-3-acetic acid), IBA (indolebutyric acid), phenylacetic acid and NAA (naphthaleneacetic acid), and mixtures thereof. Preferentially, 2,4-D (2,4-dichlorophenoxyacetic acid) is excluded from the auxins that are suitable for use in the invention, since it is an unnatural compound.

The cytokinins that are most particularly suitable for use in the invention may be chosen from kinetin (N-(furan-2-ylmethyl)-7H-purin-6-amine), zeatin (2-methyl-4-(7H-purin-6-ylamino)but-2-en-1-ol) and benzyl adenine (N-benzyl-7H-purin-6-amine), and mixtures thereof.

The gibberellins may be chosen from gibberellin A3, A1, A12, and mixtures thereof.

In step ii. of the process, the plant hormone(s) or phytohormone(s) are preferentially chosen from indole-3-acetic acid, indolebutyric acid, phenylacetic acid, naphthaleneacetic acid, kinetin, zeatin, benzyl adenine, gibberellic acid, and gibberellins A1, A3 and GA3.

In a preferred embodiment, the plant hormone(s) or phytohormone(s) are preferentially chosen from naphthaleneacetic acid and kinetin.

Preferably, the culture medium in step ii. is an aqueous medium.

For the purposes of the present invention, the term “aqueous medium” means a medium comprising water and optionally an additional aqueous solvent that is in particular compatible with the cultivation of plant cells.

According to a particular embodiment, said culture medium in step ii. is an aqueous medium comprising:
at least one plant hormone such as 1-naphthaleneacetic acid, kinetin and mixtures thereof; and
at least one salt optionally in hydrated form chosen from NH4NO3; KNO3; CaCl2 such as CaCl2.2H2O; MgSO4; KH2PO4; MnSO4 such as MnSO4.4H2O; ZnSO4 such as ZnSO4.7H2O: KI; Na2MoO4 such as Na2MoO4.2H2O; CuSO4 such as CuSO4.5H2O; Na2EDTA such as Na2EDTA.2H2O; FeSO4 such as FeSO4.7H2O; and mixtures thereof; and
at least one carbon source, preferably chosen from monosaccharides, oligosaccharides or polysaccharides, and mixtures thereof; in particular, said carbon source is chosen from glucose, fructose, sucrose, and mixtures thereof, preferably sucrose;
and optionally at least one compound chosen from myoinositol, nicotinic acid, pyridoxine HCl, thiamine HCl, and mixtures thereof;
and optionally polyvinylpyrrolidone.

Advantageously, the amount of carbon source present in the culture medium is between 5 and 40 g/l of culture medium, preferably between 10 and 30 g/l; better still, the amount of carbon source present in the culture medium is 20 g/l of culture medium.

According to a preferred embodiment, the culture medium is an aqueous medium containing at least NH4NO3; KNO3; CaCl2.2H2O; MgSO4; KH2PO4; MnSO4.4H2O; ZnSO4.7H2O; KI; Na2MoO4.2H2O; CuSO4.5H2O; Na2EDTA.2H2O; FeSO4.7H2O; myoinositol; nicotinic acid; pyridoxine HCl; thiamine HCl; naphthaleneacetic acid; kinetin; sucrose, and optionally polyvinylpyrrolidone.

Advantageously, the culture medium may comprise from 1200 to 2000 mg/l of NH4NO3; from 1500 to 2100 mg/l of KNO3; from 300 to 500 mg/l of CaCl2.2H2O; from 150 to 200 mg/l of MgSO4; from 153 to 187 mg/l of KH2PO4; from 10 to 30 mg/l of MnSO4.4H2O; from 5 to 10 mg/l of ZnSO4.7H2O; from 0.001 to 0.91 mg/l of KI; from 0.001 to 0.30 mg/l of Na2MoO4.2H2O; from 0.01 to 0.05 mg/l of CuSO4.5H2O; from 10.5 to 50 mg/l of Na2EDTA.2H2O; from 10 to 30 mg/l of FeSO4.7H2O; from 70 to 150 mg/l of myoinositol; from 0.3 to 0.6 mg/l nicotinic acid; from 0.4 to 0.6 mg/l of pyridoxine; from 0.08 to 0.15 mg/l of thiamine; from 0.001 to 11 mg/l of naphthaleneacetic acid; from 0.001 to 1 mg/l of kinetin; from 10 to 30 g/l of sucrose; and water, and optionally from 0.1 to 0.5 g/L of polyvinylpyrrolidone.

The concentrations of the various constituents included in the culture medium are expressed as mass concentrations.

The cultivation in step ii. is advantageously performed at a temperature ranging from 20 to 30° C., preferably from 24 to 28° C. and better still at 27° C.

The cultivation in step ii. is advantageously performed in a culture comprising a partial pressure of O2 of between about 8% and 80%, such as a partial pressure of O2 of 30%.

The process in step ii. may be performed via a batch, fed-batch (semicontinuous) or continuous fermentation technique, preferably a batch fermentation technique.

After cultivation in a suitable medium, the non-elicited dedifferentiated plant cells of the invention are harvested in step iii., for example by filtration, and can be lyophilized or subjected to an extraction process.

According to a particular embodiment, the cultivation step ii. is performed for a period of from 6 to 14 days, and preferentially from 6 to 10 days.

The invention also relates to the cell line isolated by the Applicant and deposited according to the Treaty of Budapest on Feb. 28, 2019, under the reference DSM 33100 with the Deutsche Sammlung von Mikroorganismen and Zellkulturen (DSMZ) [German Collection of Microorganisms and Cell Cultures].

According to the present invention, fresh or lyophilized dedifferentiated plant cells obtained in step iii. or extracts thereof recovered on conclusion of step iv., or those formulated in compositions which stabilize them, may be used.

According to a particularly preferred embodiment of the invention, an extract of non-elicited dedifferentiated plant cells may be used. The invention necessarily relates to active extracts of non-elicited dedifferentiated plant cells with regard to the effects to be obtained on improving and/or reinforcing the barrier function and/or improving moisturization. The activity of an extract of the invention may notably be evaluated by means of the various experimental protocols detailed in the examples indicated hereinbelow.

In step iv., any extraction method known to those skilled in the art may be used to prepare an extract of non-elicited dedifferentiated plant cells according to the invention. The process according to the invention may also comprise a step of adding a water-miscible organic solvent between step iii. and the extraction step iv. Step iv. may include a step of adding a water-miscible organic solvent.

As extracts that are suitable for use in the invention, mention may be made of aqueous extracts, organic extracts; or extracts obtained by mixing water with at least one organic extraction solvent that is miscible with water in all proportions such as aqueous-alcoholic extracts; said extracts optionally being in the form of dry extracts, in particular obtained by evaporation, lyophilization or atomization.

Preferably, the extracts of non-elicited dedifferentiated cells of a plant of the species Lavandula angustifolia are chosen from:
aqueous extracts of the intracellular medium, aqueous-alcoholic extracts of the intracellular medium, or organic extracts of the intracellular medium; said extracts optionally being in the form of dry extracts; or
aqueous extracts of insoluble constituents of said cells, aqueous-alcoholic extracts of insoluble constituents of said cells. or organic extracts of insoluble constituents of said cells; said extracts optionally being in the form of dry extracts; said insoluble constituents of said cells being chosen from insoluble intracellular constituents, pectocellulose walls, cell membranes, and mixtures thereof; preferably pectocellulose walls and/or cell membranes.

Even more preferably, the extracts of non-elicited dedifferentiated cells of a plant of the species Lavandula angustifolia are chosen from:
aqueous extracts of the intracellular medium, aqueous-alcoholic extracts of the intracellular medium, or organic extracts of the intracellular medium; said extracts optionally being in the form of dry extracts; or
aqueous, aqueous-alcoholic or organic extracts of pectocellulose walls and/or of cell membranes obtained by enzymatic double digestion, preferably obtained after a first step of enzymatic digestion using one or more carbohydrases, followed by a second step of enzymatic digestion using one or more proteases.

The term “aqueous extract” means an extract obtained with an aqueous extraction solvent.

The term “aqueous extraction solvent” means a solvent which is water or which consists of water.

The term “mixture of water and of at least one water-miscible organic solvent” means a water/organic solvent mixture in all proportions.

The term “aqueous-alcoholic extract” means an extract obtained with a mixture of water and ethanol in all proportions.

The term “organic extract” means an extract obtained with an organic extraction solvent.

Among the water-miscible organic extraction solvents, mention may be made of ethanol, isopropanol, propylene glycol, 1,3-propanediol, and mixtures thereof.

The term “dry extract” means an extract comprising less than 5% by weight of solvent, preferably less than 3% by weight of solvent, better still less than 1% by weight of solvent, and, in a particular embodiment, an extract comprising 0% of solvent. The solvent may be water, an organic solvent, or mixtures thereof. A dry extract that is suitable for use in the present invention may be obtained, for example, by lyophilization, atomization or evaporation.

In a first embodiment, an extraction method that is suitable for obtaining an extract according to the invention may comprise:

i. a first step of splitting the non-elicited dedifferentiated plant cells in an extraction solvent, said extraction solvent being chosen from aqueous and organic extraction solvents or mixtures of water and of at least one water-miscible organic solvent; said first step of splitting being performed, for example, using a high-pressure homogenizer, in particular at room temperature,
ii. a second step of removing the constituents in suspension of said cells so as to recover the enriched extraction solvent obtained from the first step, preferably by centrifugation followed by a filtration step.

This extraction method in particular leads to an extract of the intracellular medium of the non-elicited dedifferentiated plant cells of a plant of the species Lavandula angustifolia.

The term “constituents in suspension of said non-elicited dedifferentiated plant cells of a plant of the species Lavandula angustifolia” means the constituents that are insoluble at a temperature of 25° C. in the extraction solvent of step i.; these constituents may in particular be insoluble intracellular constituents, pectocellulose walls, cell membranes, and mixtures thereof.

The term “enriched extraction solvent” means an extraction solvent comprising intracellular constituents that are soluble at a temperature of 25° C. of non-elicited dedifferentiated plant cells of a plant of the species Lavandula angustifolia.

Step i. of splitting the non-elicited dedifferentiated cells may be performed via any technique known to those skilled in the art, for instance the use of ultrasound, or increasing the temperature to produce heat-induced splitting, or the use of mechanical constraints on the cells such as shear, the use of ultrasound or the application of a high pressure. Preferably, the splitting of the cells leading to an extract of the invention is performed by applying a high pressure, preferably a pressure of between 500 bar and 2000 bar, better still between 1000 bar and 2000 bar, notably using a high-pressure homogenizer.

When, in step i., the extraction solvent is a mixture of water and of at least one water-miscible organic extraction solvent, said organic extraction solvent may be ethanol or 1,3-propanediol. Preferably, the [water/organic extraction solvent] mass ratio is between1/2 and 1/1. In addition, the [cells/aqueous+organic extraction solvents] mass ratio is between 1/2 and 0.9/1. In addition, a step of drying the extract may be performed on conclusion of step ii., notably by concentrating to dryness, in particular using a rotary evaporator, optionally followed by resuspending in an aqueous solvent, and/or optionally followed by a lyophilization step.

When, in step i., the extraction solvent is an organic extraction solvent, said organic extraction solvent may be ethanol or 1,3-propanediol. In addition, the [cells/organic extraction solvents] mass ratio is between 1/2 and 0.9/1. In addition, a step of drying the extract may be performed on conclusion of step ii., notably by concentrating to dryness, in particular using a rotary evaporator, optionally followed by resuspending in an aqueous solvent, optionally followed by a lyophilization step.

Step ii. of removing the constituents in suspension of said cells in suspension may be performed via any technique known to those skilled in the art, preferably by a step of centrifugation preferably between 6000×G and 12 000×G, better still between 8000×G and 10 000×G, followed by filtration of the supernatant.

The centrifugation may be performed for 20 minutes to 40 minutes.

The centrifugation may be performed at a temperature of 4° C.

The filtration may be performed by means of any filtration method known to those skilled in the art, preferably using a cellulose filter, in particular a filter of between 0.1 μm and 1 μm, such as 0.7 μm, notably a 0.7 μm Whatman filter.

Said extraction method may also comprise an optional step of sterilizing the enriched extraction solvent obtained from the second step, for example by autoclaving between 115 °C and 130 °C, in particular at 121° C.

In a first variant, irrespective of the extraction solvent(s) used, the non-elicited dedifferentiated plant cells of a plant of the species Lavandula angustifolia used in step i. are fresh cells, i.e. cells which have not undergone a drying step prior to step i., notably by evaporation, lyophilization or atomization.

In a second variant, irrespective of the extraction solvent(s) used, the non-elicited dedifferentiated plant cells of a plant of the species Lavandula angustifolia used in step i. are cells which have undergone a drying step prior to step i., notably by evaporation, lyophilization or atomization.

In a second embodiment, another extraction method that is suitable for obtaining an extract according to the invention may comprise, instead of the abovementioned second step ii., a second step of removing the enriched extraction solvent obtained from the first step so as to recover the constituents in suspension of said cells, for example by centrifugation under the conditions as described above.

This second step may also be followed by the following steps:

i. optionally adjusting the pH;
ii. performing a first enzymatic digestion of the constituents in suspension of said cells;
iii. optionally adjusting the pH;
iv. performing a second enzymatic digestion of the constituents in suspension of said cells; said second enzymatic digestion being performed with one or more enzymes other than those used for said first enzymatic digestion;
v. inactivating the enzymatic digestion;
vi. optionally adjusting the pH;
vii. optionally clarifying by centrifugation;
viii. optionally lyophilizing or atomizing the supernatant obtained from step vii.

In a preferred embodiment, the constituents in suspension of said cells are subjected to a centrifugation step prior to the double digestion step and the double digestion step is performed on the pellet obtained from the centrifugation.

The pH adjustment that may precede the step of enzymatic double digestion is optional. When it is performed, the pH is modified by adding an aqueous solution of an organic or mineral acid or base, the aim of this adjustment being to adjust the pH of the pellet if need be to a value corresponding to the optimum working pH of the enzymes used in the digestion step.

Preferably, the pH adjustment is performed. Preferably, the adjustment is performed by adding a solution of an organic or mineral acid, preferably to a pH of 3 to 6, more preferentially of 3.5 to 4.5, particularly to a pH of 4. Preferably, the pH is adjusted with a citrate/phosphate buffer at a concentration of between 10 and 100 mM such as 50 mM or any other composition making it possible to buffer to the preferential pH of carbohydrases, which is between 3 and 6, and preferably to 4. The proportion of constituents in suspension of said cells/buffered solution is between 2/100 and 30/100, preferably 10/100.

Alternatively, the adjustment is performed by adding a solution of an organic or mineral base, preferably to a pH of 6.5 to 8.5, more preferentially of 7.5 to 8.5, particularly to a pH of 8. Preferably, the pH is adjusted with a potassium hydroxide or sodium hydroxide solution at a concentration of between 0.1 M and 3 M, preferably 1 M, or any other composition making it possible to buffer to the preferential pH of proteases, which is between 6.5 and 8.5, and preferably to 8. The proportion of constituents in suspension of said cells/buffered solution is between 2/100 and 30/100, preferably 10/100.

In a particular embodiment, the enzymatic double digestion of the constituents in suspension of said cells is performed using carbohydrase and protease enzymes.

According to a first variant, the digestion is, after optional adjustment of the pH to a given value, performed sequentially by the action of a carbohydrase enzyme and then by the action of a protease enzyme.

According to a second variant, the digestion is, after optional adjustment of the pH to a given value, performed sequentially by the action of a protease enzyme and then by the action of a carbohydrase enzyme.

According to one embodiment, an inactivation step, particularly thermal inactivation, takes place between the two enzymatic digestion steps. According to a first variant of this embodiment, the optionally centrifuged constituents in suspension of said cells are optionally adjusted to a given pH and then first treated with a protease enzyme, and are then subjected to an inactivation step, and then treated with a carbohydrase enzyme. According to a second variant of this embodiment, the optionally centrifuged constituents in suspension of said cells are optionally adjusted to a given pH and then first treated with a carbohydrase enzyme, and are then subjected to an inactivation step, and then treated with a protease enzyme.

According to a preferred form of the invention, the term “carbohydrase enzyme” means enzymes such as cellulases (endo-glucanases, cellobiohydrolases, beta-glucosidases), hemicellulases, xylanases, pectinases, and mixtures thereof, for instance the enzyme Viscozyme L® sold by the company Novozyme. Viscozyme L is a mixture of carbohydrases isolated from Aspergillus sp. The amount of carbohydrase used is from 0.01% to 5% by weight, more preferentially between 2% and 3% by weight, such as 2.5%. The working temperature is from 40 °C. to 60° C., preferentially 50° C. the working pH is between 3 and 6 (limits included), preferably 4, and the treatment time is from 30 minutes to 24 hours, preferentially 90 minutes, in particular with stirring from 50 rpm to 250 rpm, such as 150 rpm. The proportion of enzymatic solution/buffered suspension of constituents of said cells ranges from 0.5/100 to 20/100, preferably 2.5/100.

The term “protease enzyme” means, for example, enzymes of the classification EC3.4, such as exoproteases, endoproteases, and mixtures thereof.

According to a preferred form of the invention, the term “protease enzyme” means the enzyme Alcalase 2.4 L sold by the company Novozyme. Alcalase 2.4 L is a broad spectrum endoprotease, purified and isolated from Bacillus licheniformis. The amount of protease used is from 0.01% to 5% by weight, more preferentially between 2% and 3% by weight, such as 2.5%. The working temperature is from 40° C. to 60° C., preferentially 50° C.; the working pH is between 6.5 and 8.5 (limits included), preferentially from 7.5 to 8.5, such as 8, and the treatment time is from 30 minutes to 24 hours, preferentially 90 minutes. in particular with stirring from 50 rpm to 250 rpm, preferably 150 rpm. The proportion of enzymatic solution/buffered suspension of constituents of said cells ranges from 0.5/100 to 20/100, preferably 2.5/100.

According to a preferred embodiment, the digestion of the constituents in suspension of said cells is performed sequentially by the action of Viscozyme sold by the company Novozyme and then by the action of Alcalase 2.4 L sold by the company Novozyme.

According to a particular embodiment, the inactivation of the enzymatic digestion is thermal inactivation that may be performed over a period of between 10 minutes to 30 minutes, preferably a period of 15 minutes.

In a particular embodiment, the inactivation of the enzymatic digestion is performed at a constant temperature of between 80° C. and 90° C., preferably a temperature of 90° C.

The term “about 15 minutes” means a period of 15 min±5 min.

In a particular embodiment, at the end of the inactivation step, the pH of the reaction medium is adjusted to 7 with an organic or mineral acid or an organic or mineral base, preferably an organic or mineral acid.

The hydrolysis product obtained from the enzymatic double digestion may undergo a step of centrifugation in particular at 10 000×G, at 4° C. for 30 minutes. The supernatant obtained following this centrifugation step is dried via any drying technique known to those skilled in the art, preferably by lyophilization or atomization.

The extract thus obtained may also be called a hydrolyzate of the insoluble constituents of said cells, in particular a hydrolyzate of the pectocellulose walls and/or cell membranes.

Another extract that is suitable for use in the invention may be a dry extract of non-elicited dedifferentiated plant cells of a plant of the species Lavandula angustifolia, in particular obtained from extracts such as those mentioned in the first and second embodiments above, according to any conventional drying method such as evaporation, lyophilization or atomization. A powder is thus obtained, which may be used directly or else mixed in an appropriate solvent before use.

The non-elicited dedifferentiated plant cells of the invention, or extracts thereof, may also be used in the form of a lyophilizate. Such a lyophilizate may be obtained by any lyophilization method known to those skilled in the art.

In principle, lyophilization consists in removing the water from a liquid, pasty or solid product, by means of the combined action of cold and vacuum. When water in the solid state is heated at very low pressure, the water sublimates, i.e. it goes directly from the solid state to the gaseous state. The water vapor (or vapor of any other solvent) leaves the product and it is captured by freezing using a condenser, or trap. This technique makes it possible to preserve both the volume and the appearance of the treated product. This technique may be performed using a lyophilizer.

Lyophilization includes at least two steps: freezing, sublimation and optionally secondary desiccation.

Freezing consists in very rapidly bringing a substance to a temperature of between −20° C. and −80° C., so as to block the water in the form of ice in the situation where it was in the liquid state.

Sublimation consists in eliminating the “free” water. Under a vacuum in the region of 100 μbar to 1000 μbar, but which can vary greatly from one product to another, heat is supplied to the product; the ice undergoes sublimation. Depending on the product and the production needs, the temperature can be varied during the cycle. The water vapor is captured by a “trap” or “condenser” and the dehydration of the product proceeds continuously. When the majority of the water has undergone sublimation, the product has lost about 80% to 90% of its water.

Secondary desiccation consists in removing the captive water from the product. In this step, the vacuum is high, in the region of 5 μbar to 100 μbar. Following this step, the product is dry, in particular between 90% and 99%, such as 95%.

For example, after recovering the cells from the culture medium by filtration through a gauze of controlled porosity (about 50 μm), the cells are frozen at low temperature. preferably from −20° C. to −80° C. The frozen cells are then subjected to a step of sublimation of the ice under a vacuum ranging from 100 to 1000 μbar, and then to a step of secondary desiccation under a vacuum ranging from 5 μbar to 100 μbar.

The lyophilized non-elicited dedifferentiated plant cells may be supplemented with water or a mixture containing water before use.

### Cosmetic Composition

Advantageously, said non-elicited dedifferentiated plant cells, and/or extracts thereof, are used in an amount representing from 0.01% to 40% by weight of solids relative to the total weight of the composition containing them, and preferentially in an amount representing from 0.01% to 20% by weight of solids relative to the total weight of the composition, preferably from 0.01% to 10% by weight of solids relative to the total weight of the composition.

The composition according to the invention contains a physiologically acceptable medium.

This physiologically acceptable medium may more particularly consist of water and optionally of a physiologically acceptable organic solvent chosen, for example, from lower alcohols including from 1 to 8 carbon atoms and in particular 1 to 6 carbon atoms, for instance ethanol, isopropanol, propanol or butanol; polyethylene glycols containing from 6 to 80 ethylene oxide units; polyols, for instance propylene glycol, isoprene glycol, butylene glycol, glycerol, sorbitol or 1,3-propanediol.

It may also be an anhydrous medium, notably an oily medium containing oils and/or fatty substances other than oils.

When the physiologically acceptable medium is an aqueous medium, it has a pH that is compatible with the skin, preferably ranging from 3 to 8 and better still from 4 to 7.

When the composition includes an aqueous or aqueous-alcoholic medium, it is possible to add a fatty (or oily) phase to this medium.

The composition according to the invention is in particular a composition intended for topical application to the skin.

Thus, the compositions according to the invention containing the dedifferentiated plant cells of a plant of the species Lavandula angustifolia, or extracts thereof, as defined above may be in any presentation form conventionally used for topical application and notably in the form of aqueous, aqueous-alcoholic or oily solutions, oil-in-water (O/W), water-in-oil (W/O) or multiple (triple: W/O/W or O/W/O) emulsions, of aqueous or oily gels, of liquid, pasty or solid anhydrous products. or of dispersions of a fatty phase in an aqueous phase using spherules. these spherules possibly being polymeric nanoparticles, such as nanospheres and nanocapsules, or lipid vesicles of ionic and/or nonionic type. These compositions are prepared according to the usual methods.

In addition, the compositions used according to the invention may be more or less fluid and may have the appearance of a white or coloured cream, a pomade, a milk, a lotion, a serum, a paste or a mousse. They may be optionally applied to the skin in aerosol form. They may also be in solid form, for example in the form of a stick.

When the composition used according to the invention includes an oily phase, it preferably contains at least one oil. It may also contain other fatty substances.

As oils that may be used in the composition of the invention, examples that may be mentioned include:
hydrocarbon-based oils of animal origin; hydrocarbon-based oils of plant origin,
synthetic esters and ethers, notably of fatty acids, for instance oils of formulae R1COOR2 and R1OR2 in which R1 represents a fatty acid residue including from 8 to 29 carbon atoms and R2 represents a branched or unbranched hydrocarbon-based chain containing from 3 to 30 carbon atoms,
linear or branched hydrocarbons, of mineral or synthetic origin,
fatty alcohols containing from 8 to 26 carbon atoms,
fluoro oils which are partially hydrocarbon-based and/or
silicone oils such as volatile or nonvolatile polymethylsiloxanes (PDMS) bearing a linear or cyclic silicone chain, which may be liquid or pasty at room temperature,
and mixtures thereof.

In the list of oils mentioned above, the term “hydrocarbon-based oil” means any oil predominantly including carbon and hydrogen atoms, and possibly ester, ether, fluoro, carboxylic acid and/or alcohol groups.

The other fatty substances that may be present in the oily phase are, for example, fatty acids including from 8 to 30 carbon atoms, waxes, silicone resins and silicone elastomers.

These fatty substances may be chosen in a varied manner by a person skilled in the art in order to prepare a composition having the desired properties, for example in terms of consistency or texture.

According to a particular embodiment of the invention, the composition according to the invention is a water-in-oil (W/O) or oil-in-water (O/W) emulsion, and more particularly an O/W emulsion. The proportion of the oily phase of the emulsion may range from 5% to 80% by weight and preferably from 5% to 50% by weight relative to the total weight of the composition. The oils, emulsifiers and coemulsifiers used in the composition in emulsion form are chosen from those conventionally used in cosmetics or dermatology. The emulsifier and the coemulsifier are generally present in the composition in a proportion ranging from 0.3% to 30% by weight and preferably from 0.5% to 20% by weight relative to the total weight of the composition. The emulsion may also contain lipid vesicles.

The emulsions generally contain at least one emulsifier chosen from amphoteric, anionic, cationic or nonionic emulsifiers, used alone or as a mixture. The emulsifiers are chosen in an appropriate manner according to the emulsion to be obtained (W/O or O/W emulsion).

The cosmetic composition of the invention may also contain adjuvants that are common in the cosmetic field, such as hydrophilic or lipophilic gelling agents or thickeners, such as xanthan gum, hydrophilic or lipophilic active agents, preserving agents, antioxidants, solvents, fragrances, fillers, screening agents, odor absorbers, dyestuffs and salts. The amounts of these various adjuvants are those conventionally used in the field under consideration, for example from 0.01% to 20% of the total weight of the composition. Depending on their nature, these adjuvants may be introduced into the fatty phase, into the aqueous phase and/or into lipid spherules.

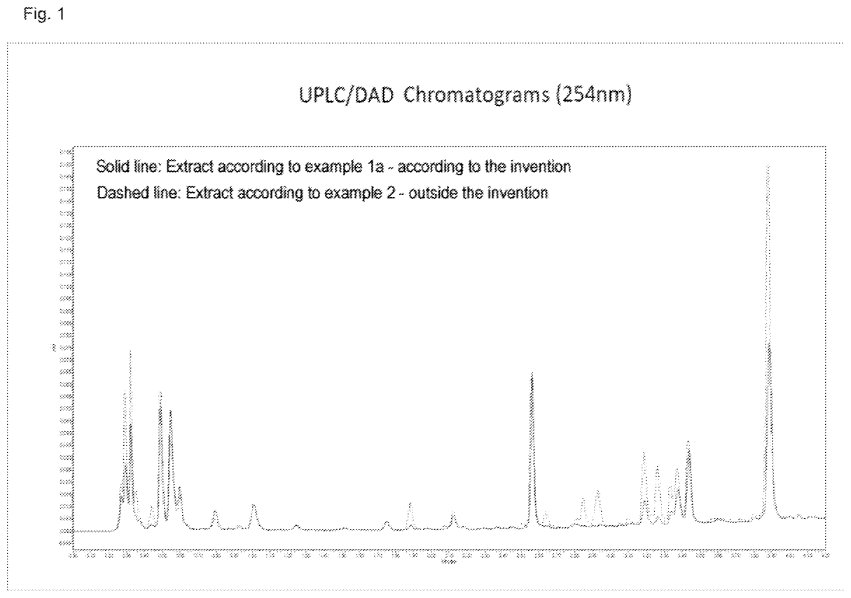

### FIGURES

[FIG. 1] HPLC chromatogram of the extract of non-elicited dedifferentiated plant cells of the species Lavandula angustifolia obtained according to Example 1a (according to the invention) versus the extract of UV-elicited dedifferentiated plant cells of the species Lavandula angustifolia obtained according to example 2 (outside the invention).

### EXAMPLES

### Example 1

Example 1a—Production of an aqueous extract of the intracellular medium of non-elicited dedifferentiated cells of Lavandula angustifolia (cultivated in Drôme, France)—according to the invention

Collection and then decontamination of the aerial parts in calcium hypochlorite (50 g/l containing 60% of active chlorine or 40 g/l) for 30 minutes. Successive rinsing in three baths of sterile osmosed water (5 minutes/bath).

Chopping of the aerial parts into explants, collecting only the leaves.

The leaves are cultivated on the culture medium shown in table 1 below on agar in light and darkness.

TABLE 1

| COMPOSITION OF THE CULTURE MEDIUM | mg/l |
|---|---|
| NH₄NO₃ | 1650 |
| KNO₃ | 1900 |
| CaCl₂•2H₂O | 440 |
| MgSO₄ | 180.8 |
| KH₂PO₄ | 170 |
| MnSO₄•4H₂O | 22.3 |
| ZnSO₄•7H₂O | 8.6 |
| KI | 0.83 |
| Na₂MoO₄•2H₂O | 0.25 |
| CuSO₄•5H₂O | 0.025 |
| Na₂ EDTA•2H₂O | 37.3 |
| FeSO₄•7H₂O | 27.8 |
| Myoinositol | 100 |
| Nicotinic acid | 0.5 |
| Pyridoxine HCl (B₆) | 0.5 |
| Thiamine HCl (B₁) | 0.1 |
| PVP (polyvinylpyrrolidone) | 200 |
| Naphthaleneacetic acid | 1 |
| Kinetin | |
| Sucrose | 20 000 |
| Water | qs 1 L |

Following the successive subculturings in the presence of culture medium, dedifferentiated plant cells which can be cultivated in a fermenter were obtained. The parameters used for the controlling the cultivation in a bioreactor are the following:
T°: 27° C.;
Aeration: pO₂ 30%, regulated by the sterile air entering and/or via stirring while avoiding any shear stress on the cells.
Stirring: 150 rpm.

The batchwise production lasts about 10 days. The culture medium obtained is then separated from the dedifferentiated cells by filtration through a gauze with a porosity of 50 μm.

On conclusion of the filtration step, said cells obtained are milled using a high-pressure homogenizer (2000 bar) in the presence of water in a [cells/water] mass ratio of 1/1. The particles in suspension are then removed by filtration at 10 000×G for 30 minutes at 4° C. followed by filtration of the supernatant using a 0.7 μm Whatman cellulose filter, so as to obtain an aqueous extract of the intracellular medium.

The aqueous extract thus obtained is dried by lyophilization under the following conditions: step of freezing the sample at −40° C., followed by sublimation by placing under vacuum (<1 mbar) at 20° C., and then secondary desiccation achieved at a lower pressure of 100 μbar by means of elimination of air injection into the system; leading to a dry extract.

Example 1b—Production of an alcoholic extract of the intracellular medium of non-elicited dedifferentiated cells of Lavandula angustifolia—according to the invention

On conclusion of the filtration step through a 50 μm gauze in example 1, said cells obtained are milled using a high-pressure homogenizer (2000 bar) in the presence of ethanol: the [cells/solvent] ratio is 1/1. The particles in suspension are then removed by filtration at 10 000×G for 30 minutes at 4° C. followed by filtration of the supernatant using a 0.7 μm Whatman cellulose filter, so as to obtain an alcoholic extract of the intracellular medium.

The extract thus obtained is dried in two steps:
by concentrating on a rotary evaporator at 50° C. followed by redissolving in water; and then
by lyophilization under the following conditions: step of freezing the sample at −40° C., followed by sublimation by placing under vacuum (<1 mbar) at 20° C., and then secondary desiccation achieved at a lower pressure of 100 μbar by means of elimination of air injection into the system; leading to a dry extract.

Example 1c—Production of a hydrolyzate of pectocellulose walls and of membranes of non-elicited dedifferentiated cells of Lavandula angustifolia—according to the invention

On conclusion of the filtration step through a 50 μm gauze in example 1, said cells obtained are milled using a high-pressure homogenizer (2000 bar) in the presence of water in a [cells/water] mass ratio of 1/1. The particles in suspension or debris are then recovered by centrifugation at 10 000×G for 30 minutes at 4° C.

A first step of enzymatic hydrolysis of the pellet obtained is performed with carbohydrases, this step enabling the hydrolysis of the cellulose compounds:
suspension of the wall debris in a solution buffered at pH 4 (50 mM citrate/phosphate buffer) to a debris/buffered solution proportion of 10/100.
hydrolysis of the wall debris by adding an enzymatic solution of carbohydrases (Viscozyme L from Novozyme) in an enzymatic solution/buffered debris suspension proportion ranging from 2.5/100. This mixture is placed at the optimum working temperature of the carbohydrases, at 50° C., and with stirring at 150 rpm for a period of 90 minutes.

A second step of protein hydrolysis with proteases is then performed:
adjustment of the pH of the reaction mixture to the preferential pH for proteases, at 8, by simple addition of a concentrated basic solution such as potassium hydroxide.
hydrolysis of the wall debris by adding an enzymatic solution of protease (Alcalase 2.4 L from Novozyme) in an enzymatic solution/buffered debris suspension proportion ranging from 2.5/100. This mixture is placed at the optimum working temperature of the proteases, at 50° C., and with stirring at 150 rpm for a period of 90 minutes.

To complete these hydrolysis steps, the hydrolysis product undergoes inactivation of the enzymes at 90° C. for 15 minutes. followed by separation of the remaining particles in suspension from the hydrolysis product obtained from the preceding two hydrolysis steps by centrifugation (10 000×G, 4° C., 30 minutes).

In order to concentrate and to store the supernatant obtained from the centrifugation, it is dried by lyophilization under the following conditions: step of freezing the sample at −40° C., followed by sublimation by placing under vacuum (<1 mbar) at 20° C., and then secondary desiccation achieved at a lower pressure of 100 μbar by means of elimination of air injection into the system.

The product obtained is called the cell wall hydrolyzate; it is rich in saccharide compounds.

### Example 2—Production of an Aqueous Extract of UV-Elicited Dedifferentiated cells of Lavandula angustifolia—Outside the Invention

During their cultivation in a production medium (cf. Table 1, culture medium composition), the cell lines of Lavandula angustifolia are elicited 10 days after being inoculated, with UV light (280-400 nm) using four Philips CLEO performance sunlamps (40-0-14/2.6) placed at a distance of 50 cm, in direct lighting above the cells for 15 hours.

This elicitation means obviously does not form any impurities in the cell culture. At the end of cultivation, i.e. 24 hours after eliciting, the plant cells are filtered through a gauze with a porosity of 50 μm to remove the remaining culture medium. On conclusion of the filtration step, a fresh biomass is thus obtained; said cells obtained are milled in a high-pressure homogenizer in the presence of water at 2000 bar so as to extract the intracellular medium of the cells. The extract thus obtained is dried by lyophilization under the following conditions: step of freezing the sample at −40° C., followed by sublimation by placing under vacuum (<1 mbar) at 20° C., and then secondary desiccation achieved at a lower pressure of 100 μbar by means of elimination of air injection into the system.

### Example 3

Example 3a: analysis of the UPLC chromatographic profile of the extracts obtained according to example 1a (according to the invention) and according to example 2 (outside the invention)

A) Materials and Methods

In order to perform the pseudo-quantitative analysis of the extracts 1a and 2, they are prepared at identical concentrations and the solutions are doped with a standard absorbing in the UV range: caffeine.

The first step consists in dissolving the samples in water to give solutions at 5 g/L. The solutions thus generated are heated gently and subjected to ultrasound for 30 minutes.

The second step consists in preparing a solution of caffeine in water at a concentration of 0.17 g/L.

The final step consists in mixing 1.8 mL of sample solution (cell extracts 1a or 2) with 0.2 mL of the caffeine solution, homogenizing the mixtures and then filtering through a 0.45 μm filter and then a 0.2 μm filter.

The analysis is performed on an Acquity UPLC Additol H-Class system (Waters) comprising a diode array detector, a Corona detector (CAD) and a single quadrupole mass spectrometer.

Chromatography System
Acquity UPLC BEH Shield RP18 column
Length 50 mm
Inside diameter 2.1 mm
Column volume 0 ml
Particle diameter 1.8 μm
Mobile phase: A=Water 0.1% HCOOH; B=ACN (acetonitrile) 0.1% HCOOH
Flow rate=0.5 mL/min
Injection volume 2 μL
Temperatures: Tcolumn=30° C.; Tsamples=20°C.

Gradient 1 is shown in Table 2 below.

TABLE 2

| T (min.) | % A | % B | P (psi) |
|---|---|---|---|
| 0 | 99 | 10 | |
| 1 | 99 | 10 | |
| 5 | 70 | 30 | |
| 6.5 | 0 | 100 | |
| 7.5 | 0 | 100 | |
| 8 | 99 | 10 | |
| 10 | 99 | 10 | |

Detection
Diode array detector (DAD): the chromatogram is recorded over the 200-700 nm range.
Corona charged aerosol detector (CAD): the pressure is set at P=35.1 psi which corresponds to a nitrogen flow rate set at D=1.21 bar.
Mass spectrometer (MS): coupling of HPLC to mass spectrometry is performed with a single quadrupole mass spectrometer equipped with an electrospray ionization (ESI) source; the mass spectrometer operates simultaneously in positive and negative ionization mode over the 100-2000 amu mass range.

B) Results

The two chromatograms of the extracts 1a and 2 were superposed (see FIG. 1).

Among the 23 target compounds:
1 compound becomes depleted when the cells are elicited with UV,
6 compounds are not affected by the elicitation,
11 compounds become enriched when the cells are elicited with UV,
5 compounds are newly synthesized (not detected in the non-elicited sample, at the limits of the method).

Thus, the two extracts 1a and 2 differ from each other in their composition.

Example 3b: evaluation of the effect of the extract of non-elicited dedifferentiated cells of Lavandula angustifolia on barrier function/moisturization markers obtained according to Example 1a (according to the invention) versus an extract outside the invention of UV-elicited dedifferentiated cells of Lavandula angustifolia obtained according to example 2 (outside the invention)

A) Materials and Methods

Cytotoxicity

Normal human epidermal keratinocytes were seeded in 96-well culture plates and then cultivated at 37° C. and 5% CO2 in culture medium for 24 hours. The medium was then replaced with culture medium containing or not containing (control) the test compounds (8 concentrations tested), and the cells were then incubated for 24 hours. All the conditions were performed in n=2. At the end of the incubation, the cell viability was measured by a standard test of measuring the mitochondrial activity with Alamar Blue®.

Analysis of the Expression of the Genes Associated with the Barrier Function/Moisturization by RT-qPCT

Normal human epidermal keratinocytes (NHEK) were seeded in 48-well culture plates and then cultivated in culture medium for 3 days at 37° C and 5% CO2 with renewal of the culture medium after the first 24 hours of cultivation. At the end of the incubation, the culture medium was replaced with test medium (supplemented with 1.5 mM CaCl2) not containing or containing (control) the test compounds, and the cells were then incubated for 24 hours. All the conditions were performed in n=2.

At the end of the treatments, the culture media were removed and the cells were rinsed twice with PBS (w/o CaCl2, w/o MgCl2). The total RNA was then isolated using a magnetic bead extraction kit and according to the supplier's recommendations (MagMAXTM-96 Total RNA Isolation Kit, Ambion). The RNA quantification and the quality control thereof were analyzed by means of Labchip GX (Perkin Elmer).

The expression of the selected transcripts was analyzed by quantitative PCR in two steps. First, the cDNAs were reverse-transcribed from the RNAs using the Quantitect® Reverse transcription kit (Qiagen) and according to the supplier's recommendations. The quantitative PCR experiments were then performed using a LightCycler® 480 Real-Time PCR System in a 384-well plate (Roche) and according to the SYBR®Green (Roche) incorporation technique. The primers used are shown in Table 2 below.

TABLE 3

| | | | | | Gene | Ref. |
|---|---|---|---|---|---|---|
| | | | Genes | Abbreviation | ID | Qiagen |
| Epidermal cohesion | | Keratinocyte | Aquaporin 3 | AQP3 | 360 | QT00212996 |
| | | differentiation | | | | |
| Moisturization | Barrier | Cornified layer | Cornifelin | CNFN | 84518 | QT00208453 |
| | function | assembly | Small proline- | SPRR1A | 6698 | QT02448782 |
| | | | rich protein 1A | | | |
| | | GAG synthesis | Hyaluronic acid | HAS3 | 3038 | QT00014903 |
| | | | synthase 3 | | | |

| | | | | |
|---|---|---|---|---|
| Epidermal renewal | Heparin-binding | HBEGF | 1839 | QT01667610 |
| | EGF-like growth | | | |
| | factor | | | |

B) Results

TABLE 4

| | Level of RNA expression | |
|---|---|---|
| | following treatment of the | Level of RNA expression |
| | NHEKs with the extract | following treatment of the |
| | obtained according to | NHEKs with the extract |
| | example 1a (extract | obtained according to |
| | according to the | example 2 (extract outside |
| Markers | invention) at 0.10 mg/l | the invention) at 0.10 mg/l |
| HBEGF | 10.39 | 5.85 |
| CNFN | 10.09 | 4.52 |
| SPRR1A | 3.94 | 1.80 |
| HAS3 | 2.87 | 1.55 |

The extract of non-elicited cells obtained according to Example 1a (extract according to the invention) markedly stimulated the expression of transcripts involved in assembly of the cornified layer (SPRR1A, CNFN), keratinocyte differentiation (AQP3) and epidermal renewal (HBEGF) compared with the extract of UV-elicited cells according to example 2 (extract outside the invention).

Thus, the extract 1a of non-elicited dedifferentiated cells from Lavandula angustifolia according to the invention proves to be particularly effective in preventing and treating dehydrated skin and improving and reinforcing the barrier function.

### Example 4—Evaluation of the Moisturizing Potential on Isolated Stratum Corneum by Measurement with a Corneometer

A test was performed to evaluate the moisturizing potential of the extracts of the invention formulated in a vehicle (80%/20% water/n-propanol) in an amount of 5% by weight relative to the total weight of the composition.

The technique makes it possible to measure the dielectric capacitance of the stratum corneum (SC), which depends on the mean dielectric permittivity value of the tissue. The dielectric permittivity varies greatly with the amount of water contained in the SC.

The SC samples are conditioned at 75% relative humidity and at 25° C. before/during the measurements and the treatment. The capacitance measurement is performed using a Corneometer™ (Courage & Khazaka, Germany).

The test extracts, extracts 1a, 1b and 1c according to the invention or a moisturizing active agent such as glycerol, are dissolved in a water/n-propanol mixture (80/20) and the solution is deposited onto the SC at a rate of 10 μL/cm²followed by air-drying for a total duration of 4 hours.

A measurement is taken at T0, before the treatment. and a measurement Ttreat(4h) is taken after total drying of the treatment.

Each treatment is systematically compared with its control (vehicle) and with its T0.

Using at least two different batches of SC, four to five SC samples are measured per treatment.

The variation in the corneometer signal (HCM) after treatment is calculated first for each SC sample: DHCMi=HCMi(Ttreat)−HCMi(T0). The mean of the DHCMi(vehicle) variations is then calculated for the control samples (treated with the vehicle): this mean value is subtracted from all the DHCMi(active agent) and DHCMi(positive control) variations to correct for the systematic bias.

The following are measured for each sample i:

For the vehicle (control): DHCMi(veh)=HCMiveh(Ttreat)−HCMiveh(T0)

For the active agent: DHCMiactive agent=HCMiactive agent(Ttreat)−HCMiactive agent(T0)

For the positive control (glycerol): DHCMipositive control=HCMipositive control(Ttreat)−HCMipositive control(T0).

To correct for the systematic bias associated with the vehicle, the corrected value DHCMicorr. active agent is considered for the active agent according to: DHCMicorr. active agent=DHCMiactive agent−M(veh)

in which M(veh) corresponds to the mean of the DHCMi(veh) variations observed on the n vehicle control samples:

$$\frac{1}{n} {\sum }_{i = 1}^{n} DHCMi ( veh ) \qquad [ Math 1 ]$$

To correct for the systematic bias associated with the vehicle, the corrected value DHCMicorr. positive control is considered for the positive control according to: DHCMicorr. positive control=DHCMipositive control−M(veh)

where M(veh) is as defined previously.

The DHCMicorr. positive control and DHCMicorr. active agent values are then normalized according to the following calculation:

% norm=[(DHCMicorr. positive control)/(M(positive control)−M(veh))]+100

norm=[(DHCMicorr. active agent)/(M(positive control)−M(veh))]+100

where M(veh) is as defined previously;

where M(positive control) corresponds to the mean of the DHCMipositive control variations observed on the n positive control samples:

$$\frac{1}{n} {\sum }_{i = 1}^{n} DHCMi ( positive control ) \qquad [ Math 2 ]$$

The % norm values that were obtained are reported in the table below for the extracts according to the invention tested at 5%, compared with glycerol at 5%:

TABLE 5

| | | | Extract 1a | Extract 1b | Extract 1c |
|---|---|---|---|---|---|
| Products tested | | | according to | according to | according to |
| in a water/n- | Vehicle 80% | Glycerol | the invention | the invention | the invention |
| propanol | water/20% | at 5% by | at 5% by | at 5% by | at 5% by |
| mixture | n-propanol | weight | weight | weight | weight |
| % norm | 0% | 100% | 116% | 115% | 190% |

This test shows that the dielectric capacitance of the stratum corneum (SC) obtained with extract 1a or 1b is similar to that obtained with glycerol at the same concentration. In addition, the dielectric capacitance of the stratum corneum (SC) obtained with extract 1c is much better than that obtained with glycerol at the same concentration.

Unexpectedly, extracts 1a, 1b and 1c according to the invention allow good moisturization of the stratum corneum and thus of the skin. This moisturizing effect proves to be either similar to (extracts 1a and 1b) or greater than (extract 1c) that of glycerol.

### Example 5—Cosmetic Composition

The following composition was prepared.

TABLE 6

| Ingredients | Amount (m/m) |
|---|---|
| Extract of non-elicited dedifferentiated | 0.01 |
| cells from Lavandula angustifolia | |
| obtained according to Example 1a | |
| Xanthan gum | 0.5 |
| Preserving agents | qs |
| Water | qs 100 |

The above composition was applied to the skin to reinforce the barrier function and/or to moisturize the skin.

1. Non-elicited dedifferentiated plant cells of a plant of the species Lavandula angustifolia, or extracts thereof.

2. The cells, or extracts thereof, as claimed in claim 1, characterized in that said extracts are chosen from aqueous and organic extracts, or extracts obtained by mixing water with at least one organic extraction solvent that is miscible with water in all proportions said extracts optionally being in the form of dry extracts.

3. The cells, or extracts thereof, as claimed in claim 1, characterized in that said extracts of non-elicited dedifferentiated cells of a plant of the species Lavandula angustifolia are chosen from:
aqueous extracts of the intracellular medium, aqueous-alcoholic extracts of the intracellular medium, or organic extracts of the intracellular medium; said extracts optionally being in the form of dry extracts; or
aqueous extracts of insoluble constituents of said cells, aqueous-alcoholic extracts of insoluble constituents of said cells, or organic extracts of insoluble constituents of said cells; said extracts optionally being in the form of dry extracts; said insoluble constituents of said cells being chosen from insoluble intracellular constituents, pectocellulose walls, cell membranes, and mixtures thereof.

4. The cells, or extracts thereof, as claimed in claim 1, characterized in that they are obtained from plant material obtained from part(s) of a plant of the species Lavandula angustifolia, said plant part(s) being one or more whole organs of the plant chosen from the leaves, the stalks, the flowers, the petals, the sepals, the seeds or the roots, or one or more fragments of said organ(s) of the plant cultivated in vivo or wild.

5. The cells, or extracts thereof, as claimed in claim 1, characterized in that they are obtained from plant part(s) chosen from the leaves or fragment(s) of leaves of the plant Lavandula angustifolia.

6. The cells, or extracts thereof, as claimed in claim 1, characterized in that they are obtained via a process comprising the following steps:
i. providing one or more parts of a plant of the species Lavandula angustifolia;
ii. cultivating said plant part(s) provided in step i. in a culture medium comprising at least one plant hormone, so as to generate dedifferentiated cells; and
iii. recovering the dedifferentiated cells obtained at the end of step ii.;
iv. optionally, extracting the dedifferentiated cells recovered in step iii.;
said process not comprising a step of eliciting said dedifferentiated cells.

7. The cells, or extracts thereof, as claimed in claim 6, characterized in that said culture medium in step ii. is an aqueous culture medium comprising:
at least one plant hormone such as 1-naphthaleneacetic acid, kinetin and mixtures thereof; and
at least one salt optionally in hydrated form chosen from NH₄NO₃; KNO₃; CaCl₂ MgSO₄; KH₂PO₄; MnSO4 ; ZnSO₄; KI; Na₂MoO₄; CuSO₄; Na₂EDTA; FeSO₄ ; and mixtures thereof; and
at least one carbon source;
and optionally at least one compound chosen from myoinositol, nicotinic acid, pyridoxine HCl, thiamine HCl, and mixtures thereof;
and optionally polyvinylpyrrolidone.

8. The cells, or extracts thereof, as claimed in claim 1, characterized in that they are obtained by cultivation of plant part(s) from the species Lavandula angustifolia, in an aqueous culture medium comprising at least NH₄NO₃; KNO₃; CaCl₂.2H₂O; MgSO₄; KH₂PO₄; MnSO4.4H₂O; ZnSO₄.7H₂O; KI; Na₂MoO₄.2H₂O; CuSO₄.5H₂O; Na₂EDTA.2H₂O; FeSO₄.7H₂O; myoinositol; nicotinic acid; pyridoxine HCl; thiamine HCl; naphthaleneacetic acid; kinetin; sucrose, and optionally polyvinylpyrrolidone.

9. A cosmetic composition comprising, in a physiologically acceptable medium, said cells and/or extracts thereof as defined in claim 1.

10. The composition as claimed in claim 9, in which said dedifferentiated plant cells, and/or extracts thereof, are used in an amount representing from 0.01% to 40% by weight of solids relative to the total weight of the composition containing them.

11. A cosmetic treatment process comprising the application of the composition as defined in claim 9 to the skin for improving and/or reinforcing the cutaneous barrier function of the skin.

12. A cosmetic treatment process comprising the application of the composition as defined in claim 9 to the skin for improving the moisturization of the skin, for preventing and/or treating roughness or the microrelief and/or for improving the radiance of the complexion and/or for improving the suppleness of the skin.

13. A cosmetic treatment process comprising the application of the composition as defined in claim 9 to the skin for improving and/or reinforcing the protection of the skin against external attack.

14. A cosmetic treatment process comprising the application of the composition as defined in claim 9 to the skin for treating the cosmetic signs of skin dryness.

15. A cosmetic treatment process comprising applying to the skin non-elicited dedifferentiated plant cells of a plant of the species Lavandula angustifolia, or extracts thereof, as defined in claim 1, for improving and/or reinforcing the cutaneous barrier function of the skin.

16. A cosmetic treatment process comprising applying to the skin non-elicited dedifferentiated plant cells of a plant of the species Lavandula angustifolia, or extracts thereof, as defined in claim 1, for improving and/or reinforcing the protection of the skin against external attack.

17. A cosmetic treatment process comprising applying to the skin non-elicited dedifferentiated plant cells of a plant of the species Lavandula angustifolia, or extracts thereof, as defined in claim 1, for improving the moisturization of the skin, for preventing and/or treating roughness or the microrelief and/or for improving the radiance of the complexion and/or for improving the suppleness of the skin.

18. A cosmetic treatment process comprising applying to the skin non-elicited dedifferentiated plant cells of a plant of the species Lavandula angustifolia, or extracts thereof, as defined in claim 1, for treating the cosmetic signs of skin dryness.

19. A cosmetic treatment process comprising the application of the composition as defined in claim 10 to the skin for improving and/or reinforcing the cutaneous barrier function of the skin.

20. A cosmetic treatment process comprising the application of the composition as defined in claim 10 to the skin for improving the moisturization of the skin, for preventing and/or treating roughness or the microrelief and/or for improving the radiance of the complexion and/or for improving the suppleness of the skin.
